(19) United States
(12) Patent Application Publication
(10) Pub. No.: US 2024/0033506 A1
(43) Pub. Date: Feb. 1, 2024

(54) DETACHABLE CASING FOR COCHLEAR IMPLANT ELECTRODES AND DEPLOYMENT APPARATUS

(71) Applicants: Michael Joseph Piatt, Cincinnati, OH (US); Charles David Rike, Springboro, OH (US)

(72) Inventors: Michael Joseph Piatt, Cincinnati, OH (US); Charles David Rike, Springboro, OH (US)

(21) Appl. No.: 18/226,025

(22) Filed: Jul. 25, 2023

Related U.S. Application Data
(60) Provisional application No. 63/392,227, filed on Jul. 26, 2022.

(51) Int. Cl. A61N 1/05; A61N 1/375

(52) U.S. Cl. CPC A61N 1/0541; A61N 1/3758

(57) ABSTRACT

An apparatus configured to receive and manipulate a cochlear implant type electrode array, where at least the front portion of the electrode, includes an outer, detachable casing or tube, where the casing is attached along the circumference of main axis of an electrode array. Predetermined portions of outer removable casing are slid off and/or removed from the electrode array by the apparatus, during electrode array insertion surgery. The removable casing, temporarily encasing the electrode array, provides a variety of functions, including, imparting additional rigidity to thin, flexible type electrode arrays, straightening pre-curved electrodes, and the like. The linear structure, provided by certain embodiments, combined with the additional rigidity, provides a surgeon additional control of an electrode array during cochlea implantation surgery. The apparatus includes a predetermined means for removable casing detachment and/or removal from an electrode array, and at least one actuator configured for surgical electrode insertion.

### PRIORITY CLAIM AND RELATED APPLICATIONS

This utility application claims the benefit of priority from provisional application U.S. Ser. No. 63/392,227 filed on Jul. 26, 2022. Said application is incorporated by reference in its entirety.

### FIELD OF THE INVENTION

The present invention is directed to the area of cochlear implant type surgeries. More specifically, the present invention is directed to a detachable casing device configured to temporarily cover a cochlear implant electrode prior to insertion. There are several advantages associated with the use of a detachable or removable casing. A typical embodiment will provide a surgeon a stiffer, straighter electrode, yielding greater electrode control during insertion; also included are the corresponding apparatuses configured to receive and manipulate such novel electrode casing systems.

### BACKGROUND OF THE INVENTION

Cochlear implants systems are typically recommended in situations where hearing impaired individuals will not obtain any substantial hearing improvements with sound amplifying type hearing aids or devices. A cochlear implant system includes an implantable electrode array configured to directly stimulate the auditory nerve and is inserted into the cochlea of the patient via a delicate surgical procedure. The human cochlea is in the shape of a spiral, beginning at a base and ending at an apex, such a complex geometry, and patent to patent variations thereof, can present insertion challenges to even the best of surgeons. Because of the delicate nature of the various soft tissue structures of the inner ear, numerous types of trauma or injuries can occur during or as a result of cochlear implantation surgery.

Traumatic insertions of an electrode array can result in a variety of injuries. One common injury is damage to the sensory cells associated with the basilar membrane, which will result in a loss of residual or natural hearing. Many investigations describe hybrid hearing as highly beneficial, a condition where residual or natural hearing is substantially superimposed with electric stimulation from the implanted electrode array system. Therefore, it is highly advantageous to preserve as much residual or natural hearing as possible.

Other injuries associated with traumatic electrode insertions include damage to the spiral ganglion cells; the organ of Corti, which houses the sound detecting hair cells; mis-insertion into the scala vestibuli instead of the target scala tympani; perforation the cochlear wall; hemorrhaging; and so forth. Additionally, injuries, immediately following electrode insertion, are also of concern. For example, the unimpeded release of the electrode array leads from the surgical insertion tool, once the electrode insertion process is complete is of significance. Avoidable handling of the electrode array leads and the like, should be minimized/eliminated in order to prevent traumas or injuries linked to the inadvertent transmission of vibrational energy into the sensitive structures of the inner ear.

Cochlear implant systems include electrode arrays possessing a straight configuration, while other arrays are designed to possess a pre-curved shape. Both configurations are fabricated on a flexible type of carrier, such as a silicone polymer, which permits bending within the curved portions of the cochlea. Certain cochlear implant electrode designs, especially those that comprise a pre-curved shape, are manufactured to include a stylet channel, which positioned along the main axis of the insertable portion of the electrode array. A stylet is a substantially thin, ridged instrument, and when housed in its corresponding stylet channel, will tend to both stiffen and straighten electrode array to provide the surgeon with better control with respect to precise electrode array positioning. During one exemplary surgical technique, the electrode array is progressively slid off of the stylet during electrode array insertion into the cochlea. Unfortunately, once the stylet is removed, the corresponding imbedded stylet channel remains as a non-functional feature of the implanted electrode array, needlessly occupying volumetric real-estate within the array. Additional bulk or material must be added to electrode designs that include stylet channel type options.

Consequently, such options drive a tendency toward bulkier, less flexible electrode array designs. The general understanding associated with the reduction of trauma related injuries as it relates to electrode arrays designs corresponds to the size and flexibility of the electrode array. Present day understanding teaches that electrode array related trauma related injuries can be reduced by the introduction of thinner and more flexible arrays.

It is apparent that there are several challenges in cochlear implant arts that long for further improvements. Given our present understanding, there are several paths to optimizing implant hearing outcomes. One path is directed to maintaining the integrity of the cochlear structures, by the elimination/reduction of injury or trauma. Yet other opportunities are based on improvements directed to the advancement of electrode array design and fabrication, as well as supporting the advancement of surgical systems and techniques that further address the preservation of delicate cochlear structures from trauma type injuries.

### SUMMARY OF THE INVENTION

The present disclosure delineates improvements to cochlear implant devices, tools, and related support systems. In one aspect of the present invention, disclosed is a removable casing or tube that envelops the electrode array, configured to possess a low friction engagement condition between the casing and the electrode array to permit sliding passage of the electrode array through the casing, as well as the safe, complete removal of the casing off of the electrode array. The removable casing in cooperation with a companion surgical tool is configured for the sequential removal of the casing along the major axis of the electrode array as the array being inserted into a Cochlea. The removal of the casing is facilitated by a separator or wedge apparatus comprising the companion surgical tool.

The system can accommodate a variety of removable casing geometries or designs, including slotted casings having a linear opening along the casing's longitudinal profile or main axis; slotted casings devoid of any pre-existing opening—where the two engaging walls comprising the slot, form an elongated slot on at least a portion of the elongated slot; and casings comprising predetermined segments, designed to disconnect or break apart when encountering a separator. The removable casing is configured to temporarily impose straightening forces and stiffen the enclosed electrode array, facilitating a controlled and precise placement of the electrode by the surgeon. In general, the initial condition of the electrode array will temporarily conform to the casing's longitudinal profile. The present invention includes embodiments configured for a variety of electrode array designs, including pre-curved modiolar hugging, straight lateral wall type designs.

Accordingly, it is an object of the present invention to provide a cochlear electrode array casing system and corresponding surgical tool for maneuvering the casing system. The systems are configured to provide improved control over surgical variables associated with the insertion of an electrode array into a cochlea so to minimize implantation trauma. By way of example, but not limitation, surgical variables include electrode array insertion angle or attack angle, electrode array insertion forces with respect to implantation, insertion speed of the electrode array, and the like. The exemplary aforementioned surgical variables are understood to have a significant impact on the preservation of residual hearing, full electrode insertion, and so forth.

It is another object of certain embodiments of present invention to encourage and enable the development of thinner, more flexible electrode arrays; it is understood that the thinner and more flexible the array, greater the probability of full electrode insertion, while keeping insertion forces and trauma type injuries to a minimum. Many present-day electrode arrays reserve a portion of the electrode's volume to accommodate a stylet channel configured to accommodate a stylet straightening rod or wire, necessary in the well-known AOS (Advanced Off-Stylet) method of electrode array insertion. The present invention does not require an electrode stylet channel, although it can accommodate such a feature. It is understood that electrode arrays, that do not require the reservation of space for an electrode stylet channel, will promote the design and advancement of thinner and more flexible, trauma minimizing designs.

It is yet another object of present invention to provide a removable means for straightening and stiffening an electrode array via a removable casing, initially encasing the electrode. The present invention further provides a means for the detachment of the removable casing during electrode array insertion into a cochlea. In manual electrode array insertions sans a stylet system, the use of a removable casing enclosing the electrode streamlines the array insertion process, requiring fewer push-release cycles by the surgeon.

It is further object of the present invention to include electrode array removable casings having generally tubular geometry possessing a longitudinal profile. The removable casing is fabricated from biocompatible casing material, possessing sufficient rigidity to temporarily force the electrode array into its predetermined longitudinal shape or profile.

The removable casing incorporates an integrated parting segment along its longitudinal profile. The initial function of the integrated parting segment is to substantially confine the electrode array about its outer circumference. During electrode insertion, an additional function of the integrated parting segment is to facilitate the separation or parting of the casing about its integrated parting segment eventually facilitating its complete removal during its sliding cooperation with a separator, located on a corresponding surgical tool.

It is an additional object of the present invention to provide a surgical tool for the precise control of an electrode array with respect to parameters associated with cochlea implantation. The surgical tool includes an actuation tool support arm having a tool support receiver, which can be connected to a variety of tool actuators. The actuators are preselected to control surgical variables associated with the insertion of an electrode array into cochlea so to minimize implantation trauma. Exemplary tool actuators include mechanical systems, electromechanical systems, robotic systems, manual systems, and any combination thereof, to achieve optimal results.

It is another object of the present invention to provide a surgical tool for controlling the motion of the removable casing with respect to the electrode array. A casing actuator attached to the extraction end of the removable casing provides a sliding retraction of the removable casing from the electrode array. Exemplary casing actuators include mechanical systems, electromechanical systems, robotic systems, manual systems, and any combination thereof, to achieve optimal results.

It is yet another object of present invention to provide a means for non-traumatic release of the entire electrode array system, including electrical leads, from the surgical tool. Once the removable casing is completely disconnected from the electrode array, the surgical tool completely releases the entire electrode array system in a non-traumatic manner.

It is another object of this invention to provide a relatively simple system that is economical from the viewpoint of the manufacturer and consumer, is susceptible to low manufacturing costs regarding labor and materials, and which accordingly evokes low prices for the consuming public, thereby making it economically available to the buying public.

Whereas there may be many embodiments of the present invention, each embodiment may meet one or more of the foregoing recited objects in any combination. It is not intended that each embodiment will necessarily meet each objective. Thus, having broadly outlined the more important features of the present invention in order that the detailed description thereof may be better understood, and that the present contribution to the art may be better appreciated, there are, of course, additional features of the present invention that will be described herein and will form a part of the subject matter of this specification.

In this respect, before explaining at least one embodiment of the invention in detail, it is to be understood that the invention is not limited in its application to the details of construction and the arrangements of the components set forth in the following description or illustrated in the drawings. The present invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent construction insofar as they do not depart from the spirit and scope of the conception regarded as the present invention.

### Particular Advantages of the Invention

The present disclosure delineates an electrode array casing system configured to encase a cochlear electrode array as well as corresponding surgical tool for precision removal of the casing system and electrode array manipulation during cochlea implantation surgery. The overall system is configured to provide improved control over surgical variables associated with the insertion of an electrode array into a cochlea so to minimize implantation trauma, vital for the preservation of residual hearing. The electrode array casing system provides a removable means for stiffening and urging a flimsy electrode array to temporarily conform to the casing system's predetermined longitudinal profile.

Another advantage of the present invention is to provide a platform that encourages the advancement of low-trauma cochlear implantation systems, including the development of thinner, more flexible electrode arrays. There's a general understanding that the thinner and more flexible the electrode array, greater the probability of full electrode insertion, and keeping insertion forces and trauma type injuries to a minimum. Several present-day electrode arrays reserve a portion of the electrode's volume to accommodate a stylet channel configured to accommodate a stylet straightening device. The present invention does not require an electrode array possessing an electrode stylet channel, although it can accommodate such a feature. By providing a system that is capable of precision manipulation of electrode arrays, which do not possess a stylet channel, a portion of the hearing-impaired public will reap the benefits as thinner, more flexible, trauma minimizing designs are developed and enter the market.

### BRIEF DESCRIPTION OF THE DRAWINGS

The ensuing detailed description section makes reference to the annexed drawings. An enhanced understanding of the present invention will become evident when consideration is given to the detailed description thereof and objects other than the aforementioned become apparent. The invention will be described by reference to the specification and the annexed drawings, in which like numerals refer to like elements, and wherein:

FIG. 1 illustrates a perspective view of an assembled cased electrode system. The system is comprised of a removable casing having an open slot and an electrode array, where the casing is constrainedly jacketing an electrode array. The cased electrode system or assembly is shown mounted in a support sleeve.

FIG. 2 illustrates a cross-sectional view of a removable casing embodiment having an open slot design along its longitudinal profile.

FIG. 3 illustrates a cross-sectional view of a removable casing embodiment having an integrated parting segment having a single engagement track located in close proximity to the casing's outer surface.

FIG. 4 illustrates a cross-sectional view of a removable casing embodiment having an integrated parting segment having a single engagement track located in close proximity to the casing's inner surface.

FIG. 5 illustrates a cross-sectional view of a removable casing embodiment having an integrated parting segment having a single engagement track. The track is centrally located between the casing's inner and outer surfaces.

FIG. 6 illustrates a cross-sectional view of a removable casing embodiment having an integrated parting segment configured as a partial slit, located about the casing's outer surface, along its longitudinal profile.

FIG. 7 illustrates a cross-sectional view of a removable casing embodiment having an integrated parting segment configured as a flat surface, having a reduced thickness, located about the casing's outer surface, along its longitudinal profile.

FIG. 8 illustrates a top view of a removable casing embodiment having a longitudinal profile. Depicted is an integrated parting segment having an interlocking profile configured between the first edge profile and mating second edge profile.

FIG. 9 illustrates a top view of a removable casing embodiment having longitudinal a profile. Depicted is an integrated parting segment having a complementary mating outline configured between the first edge profile and opposing corresponding second edge profile.

FIG. 10 illustrates a side perspective view of a surgical tool embodiment.

FIG. 11 illustrates a top perspective view of a surgical tool embodiment, primarily detailing separator 96 and cooperating proximate tool elements.

FIG. 12 illustrates a perspective view of a surgical tool embodiment including a cochlear implant mounted therein.

FIG. 13 illustrates a perspective, rear view of a surgical tool embodiment, detailing casing exit port 106 located on support end 80 of support sleeve 78.

FIG. 14 illustrates a perspective, side view of an alternate embodiment surgical tool 109. The embodiment depicts actuation tool support member 76 including a removable clamp 112 disposed thereon, and stabilizing retraction guide 118.

FIG. 15 illustrates an exemplary mechanical actuator mounted on surgical tool 68. Piston based linear actuator 122 is coupled to retractor 120, which is configured to enable the extraction of removable casing 4 from electrode array 2.

FIG. 16 illustrates an exemplary electromechanical actuator mounted on surgical tool 68. Gear and/or worm drive based linear actuator 124 is coupled to retractor 120, which is configured to enable the extraction of removable casing 4 from electrode array 2.

FIG. 17 illustrates an exemplary robotic system 130 coupled to surgical tool 68. Exemplary robotic arm assembly 132 is firmly secured to actuation tool support member 76 via tool support receiver 126. Actuation tool support member 76 has substantial control over the mechanical movements of surgical tool 68, and therefore can manage a variety of critical surgical variables, primarily directed to the insertion of electrode array 2 into a cochlea.

FIG. 18 illustrates a side perspective view of an exemplary manual surgical tool 133. The navigation of the mechanical movements of surgical tool 133 controlling electrode array 2, as well as the retraction of removable casing 4 are both configured to be accomplished manually.

FIG. 19 illustrates a side perspective view of a detail corresponding to the front portion of an exemplary manual surgical tool, including cased electrode system 1 mounted therein. The illustration further depicts slotted stop 134, configured to engage both front portion of removable casing 4, and skull external wall 136.

### DEFINITIONS OF TERMS USED IN THIS SPECIFICATION

The detachable casing for cochlear implant electrodes and deployment apparatus discussed throughout this disclosure shall have equivalent nomenclature, including, but not limited to: the device, the system, the assembly, the present invention, or the invention. Additionally, the term exemplary shall possess a single meaning throughout this disclosure; wherein the sole focus is directed to serving as an example, instance, or illustration. The terms: surgeon, or surgeons shall be broadly defined as individuals working in the field pertaining to the technology disclosed herein. The terms: front, front end, or front portion shall be understood to refer to the portion of the surgical tool or attached members that face the patient during a surgical procedure.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Note that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by”, “possessing” and “having” are all to be interpreted as open-ended terms, are all considered equivalent terms, and are used interchangeably.

### PARTS/FEATURES LIST

1—cased electrode system (removable casing encasing comprising electrode array)
2—electrode array (cochlear implant use)
3—interface (between electrode array 2 and removable casing 4)
4—removable casing (electrode casing system)
6—open slot (located on removable casing)
7—casing diameter
8—support sleeve
10—major axis (of electrode array)
12—tip (of electrode array)
14—longitudinal profile (of removable casing)
16—slot width (between first slot wall and second slot wall)
18—open slot (removable casing)
20—first slot wall
22—second slot wall
24—inner volume (configured to hold/constrain electrode array)
26—single engagement track (top)
28—casing wall thickness
30—single engagement track (bottom)
32—mediate engagement track (disposed between inner wall 34 and outer wall 36)
34—casing inner wall surface
36—casing outer wall surface
38—partial slit (only penetrates a portion of wall thickness 40)
40—wall thickness
42—flat surface
44—reduced wall thickness
46—interlocking profile (integrated parting segment)
48—longitudinal profile (of casing)
50—interlocking engagement track (non-linear)
52—first profile
54—second profile
56—second casing edge
58—first casing edge
60—complementary profile (integrated parting segment)
62—non-linear engagement track
64—first profile
66—second profile
68—surgical tool (exemplary embodiment)
70—outer tube support
72—proximate end (of outer tube support 70)
74—distal end (of outer tube support 70)
76—actuation tool support member
78—support sleeve
80—support end (of support sleeve 78)
82—mediate portion (of support sleeve 78)
84—slotted end portion (of support sleeve 78)
86—adjustment tab (for specific electrode array types)
88—implant support channel
90—top end (of implant support channel 88)
92—bottom end (of implant support channel 88)
94—electrode array cable (contains electrode array leads or wires)
95—inflection point
96—separator
98—expansion zone (support sleeve support end, electrode array 2 removed from
removable casing 4)
99—sleeve opening (top opening of support sleeve 78)
100—inner surface (of cylindrical segment 101)
101—inner cylindrical segment (support sleeve, slotted end portion 84)
102—concaved surface (of implant support channel 88)
103—inner diameter
104—retraction direction
105—open configuration (of removable casing 4, electrode array released)
106—casing exit port
107—guide rod
108—vertical wall
109—surgical tool (alternate embodiment)
110—holding channel (for electrode array cable 94)
112—channel lid (simple, low vibration engagement/disengagement)
114—extraction end (removable casing)
116—casing holding mechanism
118—stabilizing retraction bar
120—retractor
122—piston based linear actuator (mechanical actuator for casing 4 extraction)
124—electromechanical linear actuator (for casing 4 extraction)
126—tool support receiver
128—engagement arm
130—robotic actuator (controlling surgical variables associated with surgical tool 68)
132—robot arm assembly
133—manual surgical tool
134—slotted stop
136—skull external wall
137—skull opening
138—manual casing-handle
140—manual tool-handle
142—slot (slotted stop 134)
143—sleeve opening (top portion of engagement sleeve)
144—centered opening
146—first side
148—second side
150—engagement sleeve
152—casing end (of removable casing 4)

### DETAILED DESCRIPTION

With reference to the drawings of the present invention, several embodiments pertaining to the detachable casing for cochlear implant electrodes and deployment apparatus systems of the present invention thereof will be described. In describing the embodiments illustrated in the drawings, specific terminology will be used for the sake of clarity. However, the invention is not intended to be limited to the specific terms so selected, and it is to be understood that each specific term includes all technical equivalents that operate in a similar manner to accomplish a similar purpose. Terminology of similar import other than the words specifically mentioned above likewise is to be considered as being used for purposes of convenience rather than in any limiting sense.

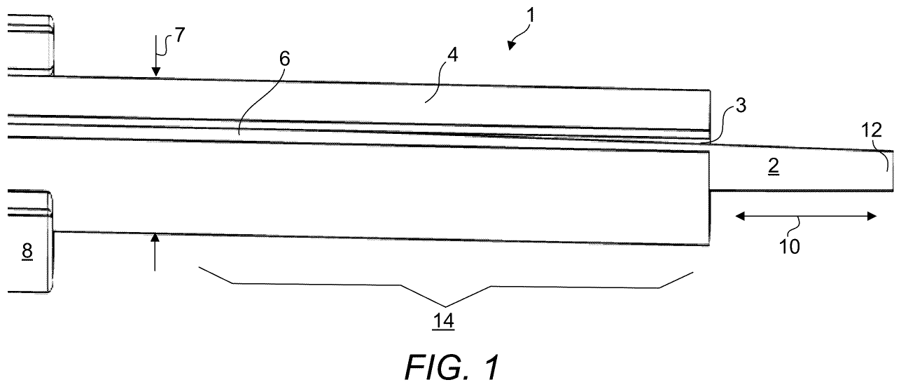

FIG. 1 illustrates a perspective view of cased electrode system 1, partially mounted in support sleeve 8. Cased electrode system 1 is composite, comprising the encasement of electrode array 2 by removable casing 4. In this figure, electrode array 2 depicted is partially encased by removable casing 4. Removable casing 4 provides a means for stiffening or constraining the lateral movements of electrode array 2. Removable casing 4 can be constructed from a wide variety of biocompatible materials. However, the material should provide sufficient rigidity such that a flexible electrode array 2 conforms to longitudinal profile 14 provided by removable casing 4, yet separate from array 2 when engaged with separator 96 and the like. An exemplary, preferred casing material includes the polypropylene family of polymers. Preferred casing material properties include transparency, to enable viewing of electrode array 2 within removable casing 4.

Interface 3 is defined as the junction or contact area where the outer surface of electrode array 2 touches inner wall surface 34. Interface 3 provides a snug, yet impermanent union between the outer surface of electrode array 2 and inner wall surface 34 of removable casing 4, providing a reliable sliding relationship between the two components. Corresponding surgical tool 68 utilizes the sliding relationship by enabling a controlled retraction of removable casing 4 from electrode array 2. The retraction of removable casing 4 by surgical tool 68 is a sub-process comprising the broader process objectives, directed to the trauma-free insertion of electrode array 2 into a cochlea. In preferred embodiments, the retraction of removable casing 4 from electrode array 2 transpires in a linear fashion. Accordingly, longitudinal profile 14 is configured in a substantially linear format, as is encased portion of electrode array 2, having major axis 10. Tip 12 of electrode array 2 is shown exposed, without removable casing 4 to drive compliance to a linear format. This unconstrained portion of electrode array 2 is disposed for insertion.

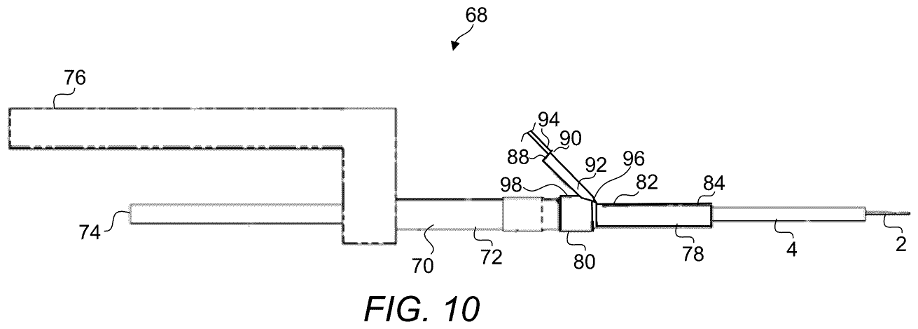

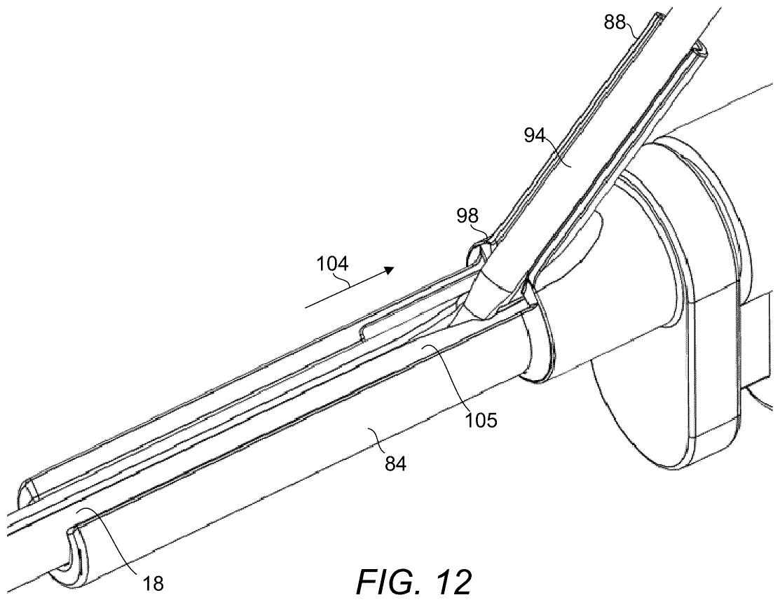

Removable casing 4 includes generally open slot 6, or slit. In this embodiment, the distance between slit walls can vary, as when a slit is formed by a sharp cutting instrument. Open slot 6 is a simple type of integrated parting segment, which is incorporated into removable casing 4. The specific features or mechanisms provided by various integrated parting segment embodiments can vary, yet all are configured to split apart removable casing 4 into an open configuration 105 (FIG. 12), subsequently facilitating the complete removal of removable casing 4 from electrode array 2 when affected by corresponding exemplary surgical tool 68—depicted in FIG. 10. Open slot 6, as with all integrated parting segment embodiments, provides a passageway or opening for electrode array cable 94 (depicted in FIG. 10) as removable casing 4 is retracted from electrode array 2 during the implantation process. Open configuration 105 (FIG. 12) provided by open slot 6, additionally furnishes a means for automatic, gentle disengagement and/or removal of electrode array cable 94 from corresponding surgical tool 68 occurring at or near the culmination of electrode array 2 cochlear insertion.

Note that for exemplary purposes, removable casings 4 are shown to possess circular cross-sectional geometries, which are configured to enclose corresponding electrode arrays 2, which have corresponding, circular cross-sectional geometries. It is understood that the cross-sectional geometries of both electrode arrays 2 and corresponding removable casings 4 can be patterned to virtually any geometric shape. In other embodiments, two different cross-sectional shapes or geometries can be used, one shape for the removable casing 4 and another for electrode array 2, and still have a situation where the removable casing 4 stiffens and constrains electrode array 2.

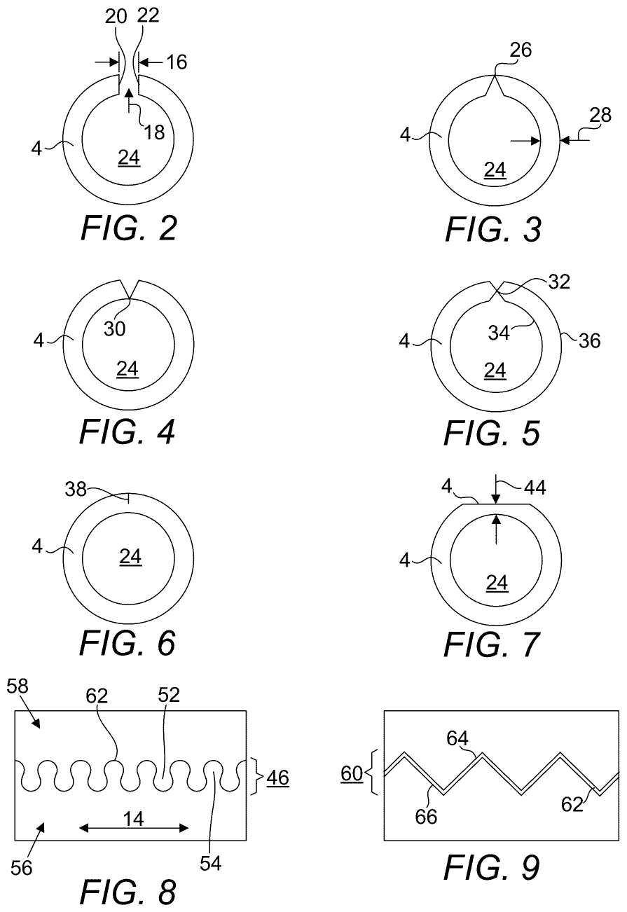

FIG. 2 illustrates a cross-sectional view of a removable casing 4 type embodiment, having an integrated parting segment configured as a uniform open slot 18. Inner volume 24 is configured to retain electrode array 2, and provides a means for stiffening and/or straightening, annexed electrode array 2. Note that the function of the integrated parting segment remains constant among all the disclosed embodiments. Open slot 18 is configured to have a predetermined, substantially constant slot width 16, established by the distance between first slot wall 20 and second slot wall 22. Open slot 18 provides a passageway or opening for electrode array cable 94 (best depicted in FIG. 12) longitudinally through removable casing 4 as removable casing 4 is retracted from electrode array 2 during implantation surgery. The passageway or opening provided by open slot 18 and the like, additionally enables a means for automatic, gentle disengagement and/or removal of electrode array cable 94 from corresponding surgical tool 68. Electrode array cable 94 disengagement occurs at or near the culmination of electrode array 2 cochlear insertion.

FIG. 3 illustrates a cross-sectional view of a removable casing 4 embodiment having an integrated parting segment having a single engagement track 26 located at the upper or top portion of removable casing 4, near or at the casing's outer surface. The single engagement track is comprised of a first edge and an opposing second edge, where the edge thicknesses are less than casing wall thickness 28. First edge and an opposing second edge substantially touch in a continuous manner; accordingly, removable casing 4 substantially seals off electrode array 2 about its longitudinal circumference. A benefit associated with using a single engagement track 26, includes additional holding power or strength removable casing 4 has upon electrode array 2. Additional holding power can prove beneficial when adjustments/corrections to electrode array 2 are made by the surgeon, which may otherwise, inadvertently, dislodge electrode array 2 from removable casing 4. All removable casing 4 embodiments of FIGS. 2-7 differ with respect to their individual integrated parting segment, which provides a means for facilitating casing 4 removal from electrode array 2; all other geometric features among the embodiments are common.

FIG. 4 illustrates a cross-sectional view of a removable casing 4 embodiment having an integrated parting segment having a single engagement track 30 located at the lower or bottom portion of removable casing 4, near or at the casing's inner wall surface 34. The single engagement track is comprised of a first edge and an opposing second edge, where the edge thicknesses are less than casing wall thickness 28. First edge and an opposing second edge substantially touch in a continuous manner; accordingly, removable casing 4 substantially seals off electrode array 2 about its longitudinal circumference. A benefit associated with using a single engagement track 30, includes additional holding power or strength removable casing 4 has upon electrode array 2. Additional holding power can prove beneficial when adjustments/corrections to electrode array 2 are made by the surgeon, which may otherwise, inadvertently, dislodge electrode array 2 from removable casing 4.

FIG. 5 illustrates a cross-sectional view of a removable casing 4 embodiment having an integrated parting segment having a mediate engagement track 32, located between casing inner wall surface 34 and casing outer wall 36. Mediate engagement track 32 is a single engagement type track comprising a first edge and an opposing second edge, where the edge thicknesses are less than casing wall thickness 28. First edge and an opposing second edge substantially continuously touch; accordingly, removable casing 4 substantially seals off electrode array 2 about its longitudinal circumference. A benefit associated with using a mediate engagement track 32, includes additional holding power or strength removable casing 4 has upon electrode array 2. Additional holding power can prove beneficial when adjustments/corrections to electrode array 2 are made by the surgeon, which may otherwise, inadvertently, dislodge electrode array 2 from removable casing 4.

FIG. 6 illustrates a cross-sectional view of a removable casing 4 embodiment having an integrated parting segment configured as partial slit 38, longitudinally disposed on casing outer wall 36, but not reaching the depth of casing inner wall surface 34. The depth of partial slit 38 is less than the casing wall thickness 28. Accordingly, casing inner wall surface 34 forms a continuous barrier which substantially seals off electrode array 2 about its longitudinal circumference. A benefit associated with using a partial slit 38, includes additional holding power or strength removable casing 4 has upon electrode array 2. Additional holding power can prove beneficial when adjustments/corrections to electrode array 2 are made by the surgeon, which may otherwise, inadvertently dislodge electrode array 2 from removable casing 4. Corresponding surgical tool 68 is configured to split apart partial slit 38, transforming removable casing 4 into open configuration 105 (FIG. 12), subsequently facilitating the complete removal of removable casing 4 from electrode array 2.

FIG. 7 illustrates a cross-sectional view of a removable casing 4 embodiment having an integrated parting segment configured as flat surface 42, longitudinally disposed on casing outer wall 36. Flat surface 42 creates reduced wall thickness 44 with respect to casing wall thickness 28. Accordingly, casing inner wall surface 34 forms a continuous barrier which substantially seals off electrode array 2 about its longitudinal circumference. A benefit associated with using flat surface 42, includes additional holding power or strength removable casing 4 has upon electrode array 2. Additional holding power can prove beneficial when adjustments/corrections to electrode array 2 are made by the surgeon, which may otherwise, inadvertently, dislodge electrode array 2 from removable casing 4. Corresponding surgical tool 68 is configured to split apart the flat surface 42 feature, transforming removable casing 4 into open configuration 105 (FIG. 12), subsequently facilitating the complete removal of removable casing 4 from electrode array 2.

FIG. 8 illustrates a top view of a removable casing 4 embodiment having an integrated parting segment configured as an interlocking profile 46, disposed along longitudinal profile 14. Interlocking profile 46 includes a non-linear interlocking engagement track 50 comprising a first casing edge 58 having a first profile 52; and a second casing edge 56 having a second profile 54. The complementary interlocking profiles created by interlocking engagement track 50 forms a continuous barrier which substantially seals off electrode array 2 about its longitudinal circumference. Additionally, the interlocking engagement scheme substantially increases the tensile strength of the junction, resulting in additional holding power or strength removable casing 4 possesses over electrode array 2. Additional holding power can prove beneficial when adjustments or corrections to electrode array 2 are made by the surgeon. For example, the prevention of unintentional dislodgement of electrode array 2 from removable casing 4. Corresponding surgical tool 68 is configured to split apart non-linear interlocking engagement track 50, transforming removable casing 4 into open configuration 105 (FIG. 12), subsequently facilitating the complete removal of removable casing 4 from electrode array 2.

FIG. 9 illustrates a top view of a removable casing 4 embodiment having an integrated parting segment 46 disposed along longitudinal profile 14. Non-linear engagement track 62 includes complementary profile 60 comprising a first profile 64; and a second profile 66. One embodiment of a complementary interlocking profile is created by the complete engagement of first profile 64; with complementary second profile 66, thereby forming a continuous barrier which substantially seals off electrode array 2 about its longitudinal circumference. Another embodiment of a complementary interlocking profile is created by the near engagement of first profile 64; with complementary second profile 66, where gap of predetermined width is produced. Because the length of non-linear engagement track 62 is substantially longer than its slotted counterpart open slot 18 of FIG. 2, removable casing 4 is provided superior holding power upon electrode array 2. Additional holding power can prove beneficial when adjustments/corrections to electrode array 2 are made by the surgeon. For example, the prevention of unintentional dislodgement of electrode array 2 from removable casing 4. Corresponding surgical tool 68 is configured to split apart non-linear interlocking engagement track 62, transforming removable casing 4 into an open configuration 105 (FIG. 12), subsequently facilitating the complete removal of removable casing 4 from electrode array 2.

FIG. 10 illustrates a side perspective view of surgical tool 68 embodiment. Preferred embodiments of surgical tool 68 are configured to receive and manipulate a variety of cased electrode systems 1. Cased electrode systems 1 are comprised of electrode array 2 with an attached removable casing 4 thereon. Surgical tool 68 is configured to facilitate the insertion of electrode array 2 into a cochlea.

Outer tube support 70 is a primary support structure comprising surgical tool 68; outer tube support 70 includes a hollow internal volume which is configured to contain or accept removable casing 4 in an open configuration 105. Actuation tool support member 76 is securely attached to distal end 74 portion of outer tube support 70; tool actuation support member 76 further includes a tool support receiver attached thereon for receiving and securing a surgical tool actuator. Proximate end 72 of outer tube support 70 is concentrically attached to support end 80 of support sleeve 78. Support end 80 of support sleeve 78 additionally includes an optional expansion zone 98, approximately located where removable casing 4 enters proximate end 72 outer support tube 70 in open configuration 105 (FIG. 12). In preferred embodiments, the diameter of expansion zone 98 is larger than the diameter of support sleeve 78, to accommodate the larger overall diameter of a removable sleeve in open configuration 105 (FIG. 12). Slotted end portion 84 of support sleeve 78 possesses an open top and is configured to support cased electrode system 1—containing removable casing 4 and electrode array 2.

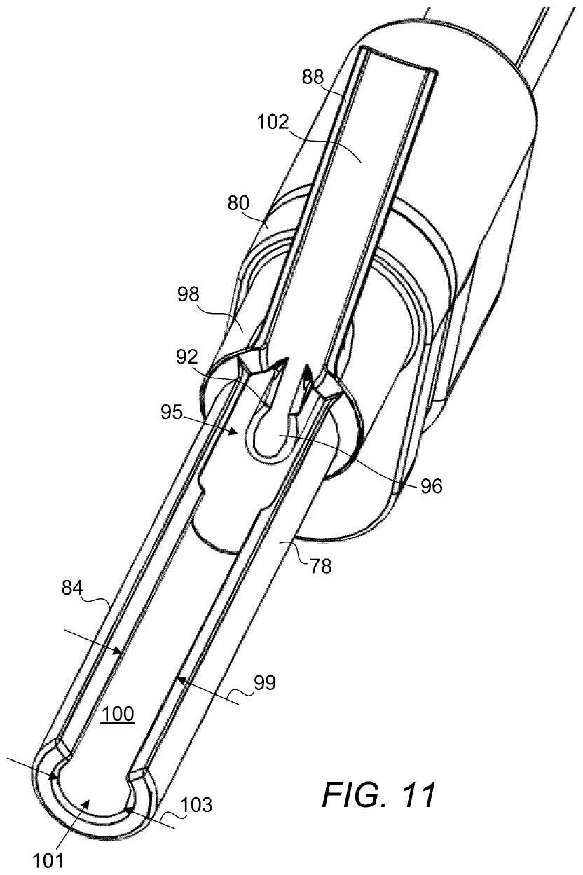

Mediate portion 82 of support sleeve 78 is attached to bottom end 92 portion of implant support channel 88 in a fish-mouth coupling arrangement-a fabrication configuration enabling the joining or coupling of two tubular structures such that the inner volumes of each tube are conjoined, substantially creating a new continuous tubular network. Implant support channel 88 and support sleeve 78 are obliquely connected forming an obtuse angle as depicted in FIGS. 10-12. The corresponding vertex of aforesaid obtuse angle is approximately located at inflection point 95—as shown in FIG. 11. Electrode array cable 94 is depicted exiting top end 90 of implant support channel 88, electrode array cable 94 includes electrical leads and the like from electrode array 2.

Separator 96 is a linear extension of implant support channel 88 seamlessly disposed at bottom end 92. Separator 96 device configured to gently disengage and extract removable casing 4 from cased electrode system 1 assembly, via a prying or wedging type engagement directed to interface 3, as well as immobilizing installed electrode array 2 at about inflection point 95 during removable casing 4 retraction process. Separator 96 additionally protects electrode array system from damaging pressures created by the surgical tool 68 during the implantation process.

FIG. 11 illustrates a top perspective view of surgical tool 68, detailing separator 96 and cooperating elements. Exemplary separator 96 is depicted having a general spoon or spade-like geometry, configured to disengage and extract removable casing 4 from electrode array 2 contained within cased electrode system 1. Separator 96 is positioned at a predetermined location at or near inflection point 95 to engage interface 3 of cased electrode system, facilitating the disengagement and extraction of removable casing 4 from electrode array 2. The continuous tubular network comprising implant support channel 88 and support sleeve 78 form a union having an obtuse angle possessing a vertex substantially coinciding with inflection point 95.

Implant support channel 88 comprises a concaved surface 102 that has an open configuration for supporting electrode array cable 94. Concaved surface 102 is configured without restraining type obstructions, enabling unencumbered removal of electrode array cable 94 from implant support channel 88. Support sleeve 78 includes inner cylindrical segment 101 having inner diameter 103 and sleeve opening 99. In preferred embodiments sleeve opening 99 is smaller than inner diameter 103 creating a means for restraining electrode array 2 inside support sleeve 78 in situations where casing diameter 7 is larger than sleeve opening 99; in other words, when the cross-section of the U-shaped inner wall of inner cylindrical segment 101 (based on the Cartesian circular coordinate system) is over 180 degrees. In some preferred embodiments casing diameter 7 of electrode array 2 approximates inner diameter 103 such that casing outer wall surface 36 substantially engages inner surface 100, yet the engagement forces must permit a sliding relationship between casing outer wall surface 36 and inner surface 100 to enable the retraction of removable casing 4 from electrode array 2 as further depicted in FIG. 12.

FIG. 12 illustrates a perspective view of surgical tool 68 including a cochlear implant system mounted therein. A mounted cochlear implant system is comprised of two key portions; cased electrode system 1—contained by inner cylindrical segment 101, and electrode array cable 94—shown resting on concaved surface 102 of implant support channel 88.

The following is an aggregate discussion highlighting fundamentals of operation with respect to the implantation system. Removable casing 4 portion of cased electrode system 1 is pulled in retraction direction 104 via retractor 120. While separator 96 immobilizes electrode array 2 at its approximate inflection point 95—which substantially corresponds to the bending point induced on electrode array 2 by implant support channel 88 and support sleeve 78 obtuse tubular configuration. Simultaneously, removable casing 4 is being forced into open configuration 105 as its being slidingly stripped off of electrode array 2, and entering expansion zone 98. As removable casing 4 is retracted, tip 12 of electrode array 2 is simultaneously ejected from removable casing 4, as depicted in FIG. 1. The retraction or pulling action on removable casing 4, results in the controlled ejection of electrode array 2, which is controlled by the surgeon, system or the like.

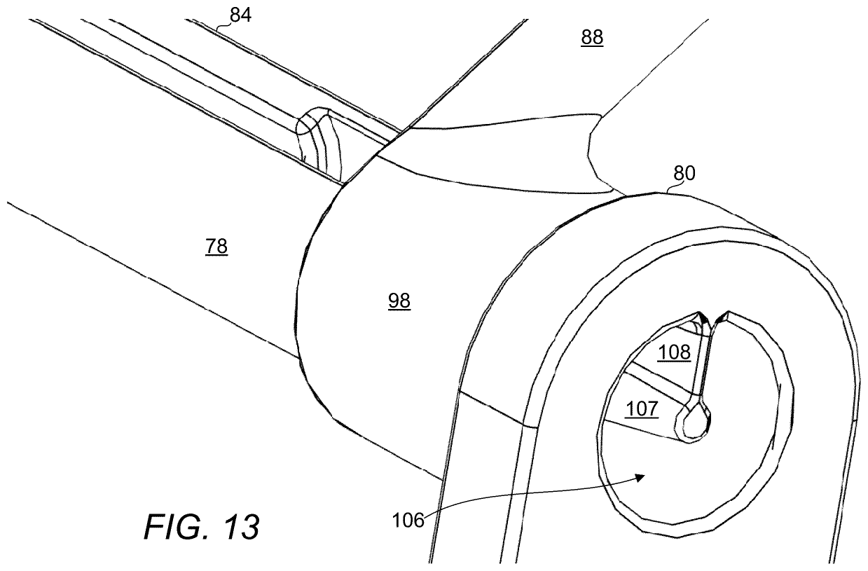

FIG. 13 illustrates a rear perspective view of surgical tool 68, detailing the support end 80 of support sleeve 78, without an outer tube support 70 element attached. Depicted is expansion zone 98 portion of support sleeve 78, comprising casing exit port 106, which expels removable casing 4, in open configuration 105 following the detachment from electrode array 2. Casing exit port 106 includes guide rod 107 supported by vertical wall 108, which provides a guide for the exiting removable casing 4. Vertical wall 108 also prevents twisting of removable casing 4 as its being retracted, providing a means for anti-rotation directed to exiting removable casing 4.

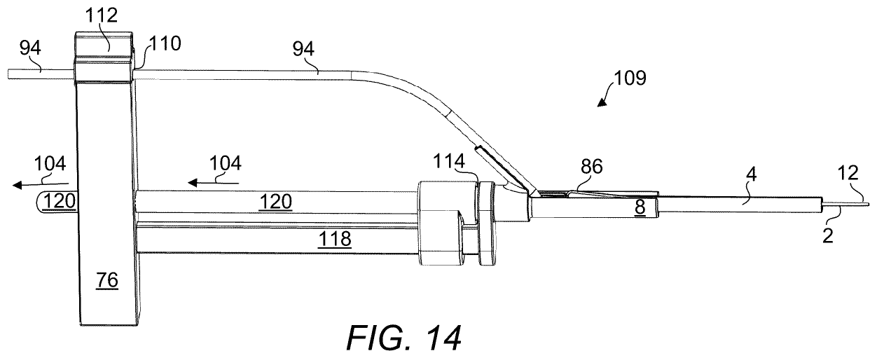

FIG. 14 illustrates a side perspective view of an alternate surgical tool 109 embodiment. Surgical tool 109 depicts actuation tool support member 76 having holding channel 110 configured to temporarily hold electrode array cable 94 to prevent snagging or pulling that can damage the implant system and/or injure a patient. A channel lid 112 is provided to further secure electrode array cable 94 onto actuation tool support member 76. Channel lid 112 provides a means for low vibration engagement/disengagement of electrode array cable 94. Preferred embodiments can use magnets to provide the holding forces necessary.

The front portion of casing holding mechanism 116 is securely attached to extraction end 114 of removable casing 4. The opposing or back end of casing holding mechanism 116 is attached to retractor 120. Lower portion of casing holding mechanism 116 is slideingly connected to stabilizing retraction bar 118—having a non-circular cross-sectional geometry, possessing anti-rotation features, to prevent twisting of removable casing 4 and the like. Retractor 120 is slideingly received via a corresponding opening located on the lower portion of stabilizing retraction bar 118. As retractor 120 is drawn in retraction direction 104, casing holding mechanism 116 and connected removable casing 4 slideingly follow in a linear manner. Accordingly, removable casing 4 is being forced into open configuration 105 as its being slidingly stripped off of electrode array 2—simultaneously tip 12 of electrode array 2 ejects from removable casing 4; thereby providing the surgeon, system or like, insertion control of electrode array 2. The figure further depicts adjustment tab 86, which is included in specific electrode array types commercially available. The present invention can accommodate such tabs, but are not required nor provide additional advantages.

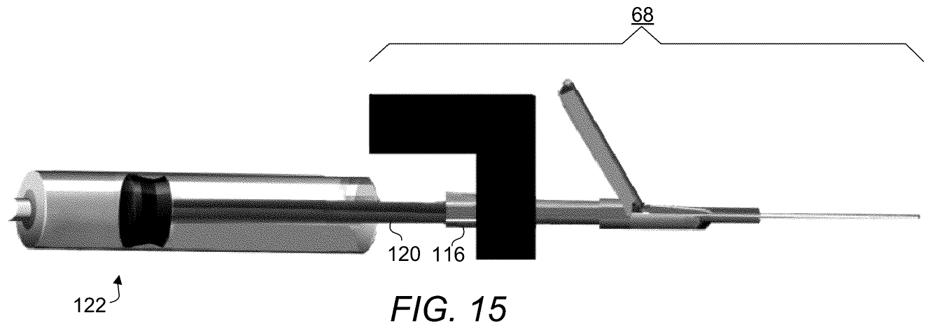

FIG. 15 illustrates an exemplary mechanical actuator mounted on surgical tool 68. Exemplary piston based linear actuator 122 is coupled to retractor 120, which is configured to enable extraction of removable casing 4 from electrode array 2. Linear piston actuators are devices used to convert fluid pressure into linear motion. They are commonly used in a variety of industrial applications. Linear piston actuators can utilize a variety of fluids, including oil, air, and the like, depending on reliability and performance desired.

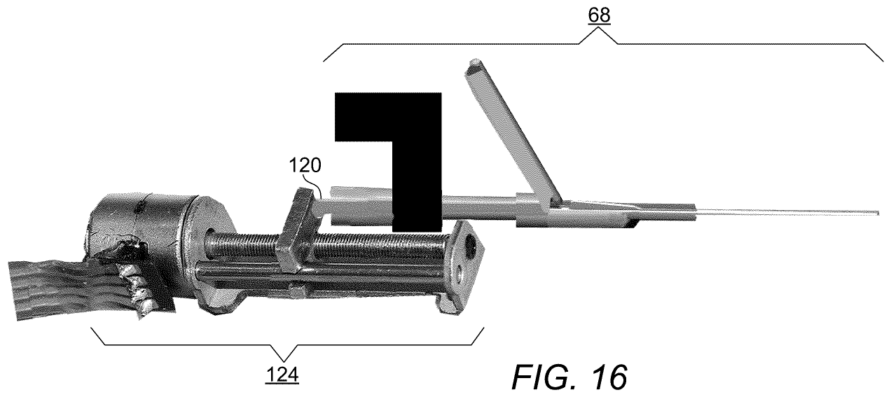

FIG. 16 illustrates an exemplary electromechanical actuator mounted on surgical tool 68. Gear and/or worm drive based linear actuator 124 is coupled to retractor 120, which is configured to power the extraction of removable casing 4 from electrode array 2. Generally, electromechanical linear actuators are comprised of a controller, an electric motor, a screw mechanism, a carriage, a transmission, and the like; or any combination thereof. Electromechanical linear actuators provide a reliable and efficient solution for converting electrical energy into linear motion, offering precise control.

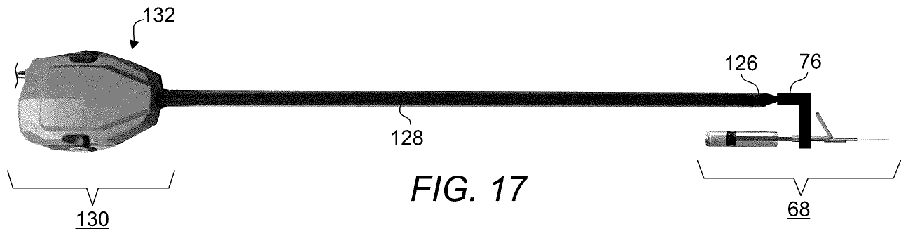

FIG. 17 illustrates an exemplary robotic system 130 coupled to surgical tool 68. Exemplary robotic arm assembly 132 in conjunction with engagement arm 128 is coupled to actuation tool support member 76 via tool support receiver 126. Actuation tool support member 76 has virtually complete control over the mechanical movements of surgical tool 68, and therefore can manage a variety of critical surgical variables directed to the insertion of electrode array 2 into a cochlea. By way of example, but not limitation, robotic controllable variables include an entry angle of electrode array 2, angle adjustments-electrode array has entered the cochlea, electrode array 2 rate of speed entry into the cochlea. Advanced electromechanical motion systems are well known in the surgical arts, present day systems are more than capable of performing the aforementioned actuation tasks associated with the manipulation of surgical tool 68 as well as the linear removal of casing 4 from electrode array 2. Such exemplary advanced electromechanical motion systems are described in U.S. Pat. No. 10,661,453 (Issued on May 26, 2020), which is herein incorporated by reference in its entirety.

In one exemplary embodiment, surgical tool actuator (e.g., system 130)—controlling surgical tool 68, in cooperation with removable casing actuator (e.g., linear system 122)—controlling the retraction rate of removable casing 4, provides a complete means for the insertion of electrode array 2 into a cochlea in accord with the aforementioned critical surgical variables. As removable casing 4 is being retracted at a predetermined rate of speed, electrode array 4 is being ejected from removable casing 4 at an identical rate of speed. In synchronization with electrode array 4 is ejection rate, surgical tool actuator (e.g., system 130) provides forward movement into a cochlea. Additionally, surgical tool actuator (e.g., system 130) will also adjust, as necessary, electrode array 4 angular considerations with respect to the cochlea to minimize cochlea trauma or injury.

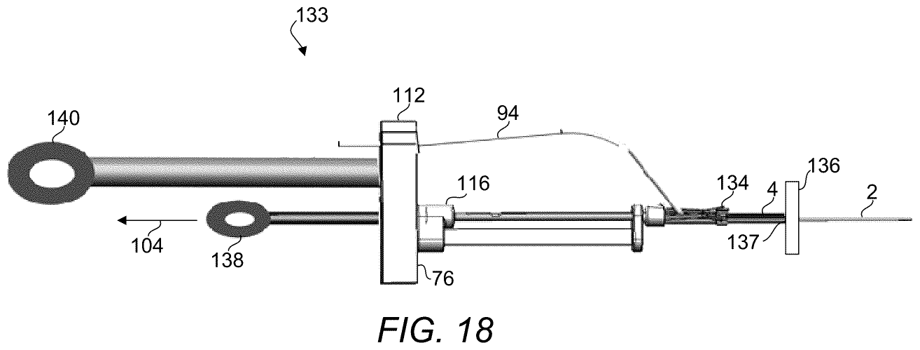

FIG. 18 illustrates a side perspective view of an exemplary manual surgical tool 133. Positional movements of surgical tool 133 controlling electrode array 2 during the cochlear implant procedure, as well as the retraction of removable casing 4 are both configured to be accomplished manually in the depicted surgical tool 133 embodiment.

The core features of manual surgical tool 133 embodiment are similar to those of embodiment surgical tool 109 shown in FIG. 14, differences include the introduction of the manual handles configured for actuations, and slotted stop 134 configured to facilitate manual insertion of electrode array 2 into a cochlea. Depicted is electrode array 2 partially inserted into the inner portion of a skull, via skull opening 137. Manual surgical tool 133 provides a surgeon with full manual control of all surgical variables.

Manual tool-handle 140 is securely affixed to actuation tool support member 76 and possesses substantial angular control of electrode array 2 housed inside removable casing 4 at or near skull opening 136. In one procedural embodiment a surgeon moves manual surgical tool 133 toward the skull, electrode array 2 enters the internal portion of the skull as removable casing 4 is retracted in a synchronized manner. Accordingly, the surgeon is in manual control of the insertion rate of speed associated with electrode array 2. It is understood that removable casing 4 provides most of the stiffness to cased electrode system 1 (removable casing 4 encasing comprising electrode array 2), and is configured to withstand all the manual forces exerted by the surgeon and still maintain its substantially linear geometry.

Once electrode array 2 insertion into the cochlea is complete, the surgeon retracts manual casing-handle 138 (functional equivalent of retractor 120 of FIG. 14) in order to fully remove removable casing 4 from electrode array 2 and permit streamlined release of any remaining portions of electrode array 2 and electrode array cable 94 from manual surgical tool 133, see FIG. 12 for supporting depiction.

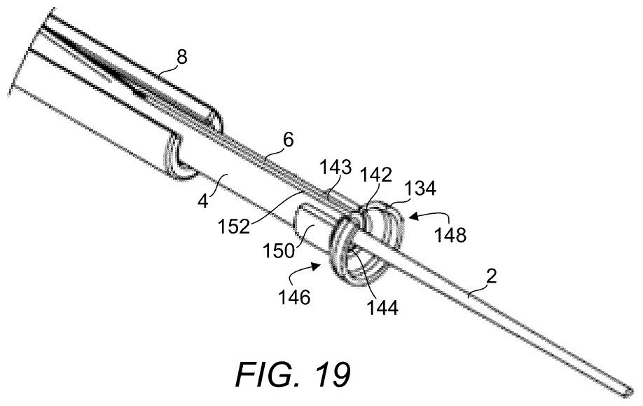

FIG. 19 illustrates details associated with the front portion of manual surgical tool 133, including cased electrode system 1 mounted therein. Depicted is slotted stop 134 having a bare electrode array 2 exiting from centered opening 144. Skull external wall 136 shown in FIG. 18, is not depicted for clarity. Support sleeve 8 is depicted retaining cased electrode system 1 comprising removable casing 4 securing electrode array 2.

Slotted stop 134 functions as a force absorbing guide, allowing a surgeon to apply force to manual surgical tool 133 via manual tool-handle 140, resulting in the insertion of electrode array 2 into a skull (cochlea) as removable casing 4 is retracted in a synchronized manner. Accordingly, the surgeon is in manual control of the insertion rate of speed associated with electrode array 2, in addition to the angular placement of manual surgical tool 133 with respect to skull opening 137. It is understood that a substantial factor driving successful cochlear implant surgery is surgeon skill and experience.

Slotted stop 134 includes first side 146 having an engagement sleeve 150 with sleeve opening 134. Engagement sleeve 150 is configured to releasably attach to casing end 152 of removable casing 4. Other embodiments of engagement sleeve 150 can be configured to permanently attach to casing end 152. Sleeve opening 143 and slot 142 are in alignment and are both configured with sufficiently large open cavities to enable unhindered passage or release of electrode array 2 and/or electrode array cable 4, each having predetermined diameters. Slotted stop 134 includes second side 148, configured to engage skull external wall 136, and a centered opening 144 for the passage of electrode array 2 through skull opening 137 and into the cochlea.

Once electrode array 2 insertion into the cochlea is deemed complete by the surgeon, retraction of manual casing-handle 138 (functional equivalent of retractor 120 of FIG. 14) will enable the complete expulsion of removable casing 4 from electrode array 2 and permit streamlined release of any remaining portions of electrode array 2 and electrode array cable 94 out of slotted stop 134 and manual surgical tool 133.

What is claimed herein is:

1. An electrode casing system adapted to releasably enclose at least a portion of an electrode array configured for implantation into a cochlea, said electrode casing system comprising:
a removable casing, comprising a generally tubular geometry having a longitudinal profile, fabricated from a casing material having sufficient rigidity such that said electrode array temporarily conforms to said longitudinal profile provided by said removable casing when encased therein; and
said casing further comprising an integrated parting segment disposed along said longitudinal profile of said casing, said integrated parting segment is configured to separate said casing into an open configuration when cooperating with a separator.

2. The electrode casing system of claim 1, wherein said longitudinal profile of said removable casing is linear.

3. The electrode casing system of claim 1, wherein said removable casing is transparent.

4. The electrode casing system of claim 1, wherein said removable casing is configured from a polymeric material.

5. The electrode casing system of claim 1, wherein said integrated parting segment is configured in an open slot configuration, said open slot configuration comprises a predetermined distance between a first slot wall and an opposing second slot wall such that said electrode casing substantially retains said electrode array incased therein.

6. The electrode casing system of claim 1, wherein said integrated parting segment comprises a first slot wall and an opposing second slot wall which substantially engage each other along at least one engagement track when constrainedly attached to said electrode array, whereby said removable casing provides a substantially gap-free enclosure surrounding the external surface of said electrode array along its major axis.

7. The electrode casing system of claim 6, wherein said at least one engagement track is linear.

8. The electrode casing system of claim 6, wherein said at least one engagement track is non-linear.

9. The electrode casing system of claim 8, wherein said at least one engagement track further comprises a set of complementary interlocking profiles, thereby increasing tensile strength.

10. The electrode casing system of claim 1, wherein said removable casing includes said electrode array slidingly attached therein.

11. A surgical tool adapted to receive and manipulate a cased electrode system, where said cased electrode system comprises an electrode array and a removable casing attached thereon; said surgical tool is configured to facilitate the insertion of said electrode array into a cochlea, said surgical tool comprising:
an outer tube support comprising a proximate end and a distal end;
an actuation tool support member affixed to a portion of said distal end of said outer tube support, said tool actuation support member further comprising a tool support receiver for the engagement of an actuator;
a support sleeve comprising a support end, concentrically attached to said proximate end of said outer tube; a slotted end portion, configured to support said cased electrode system; and a mediate portion disposed between said support end and said slotted end portion;
an implant support channel comprising a top end and a bottom end, said bottom end of said implant support channel is obliquely attached to said mediate portion of said support sleeve so to form an obtuse angle therewith, said obtuse angle having an inflection point, and the opening of said implant support channel aligns in a continuous manner with the slot on said slotted end portion of said support sleeve;
a separator, concentrically disposed at approximately said inflection point between said mediate portion of said support sleeve and said bottom end of said implant support channel, such that said separator is configured to facilitate the removal of said removable casing from said cased electrode when said separator slidingly engages with a predetermined portion of said cased electrode system.

12. The surgical tool of claim 11, wherein said surgical tool further including a casing actuator adapted to engage with said removable casing encasing said electrode array; said removable casing comprising an electrode end and an extraction end, said casing actuator includes a casing holding mechanism for securely grasping said extraction end of said removable casing such that said casing actuator provides a sliding retraction of said removable casing from said electrode array.

13. The surgical tool of claim 11, wherein said support end portion of said support sleeve further includes an anti-rotation mechanism to prevent twisting of said removable casing while said removable casing is slidingly retracted from said electrode array.

14. The surgical tool of claim 11, wherein said surgical tool further including a tool actuator adapted to engage with said tool support receiver located on said actuation tool support member; said tool actuator is configured to control surgical variables associated with the insertion of said electrode array into said cochlea so to minimize implantation trauma.

15. The surgical tool of claim 12, wherein said casing actuator is substantially fabricated from a motion system configured to provide linear motion, said motion system selected from the group consisting of a mechanical system, an electromechanical system, a robotic system, a manual system, and any combination thereof.

16. The surgical tool of claim 14, wherein said tool actuator is selected from the group consisting of a mechanical system, an electromechanical system, a robotic system, a manual system, and any combination thereof.

17. The surgical tool of claim 11, wherein said surgical tool further including a casing actuator adapted to engage with a removable casing, said removable casing comprising an electrode end, said casing actuator comprising a slotted stop; said slotted stop comprising,
a generally centered opening configured to accommodate said electrode array;
a first side, concentrically attached onto said electrode end of said removable casing;
a second side, configured to engage with an immobile portion of a patient's skull enabling the sliding removal of said removable casing from said electrode array as said surgical tool advances said electrode array into said cochlea;
a slot, configured to release said electrode array contained within said generally centered opening, once the insertion of said electrode array into said cochlea is complete.
